(19) United States
(12) Patent Application Publication
(10) Pub. No.: US 2025/0040993 A1
(43) Pub. Date: Feb. 6, 2025

(54) DETECTION OF POSITIONAL DEVIATIONS IN PATIENT REGISTRATION

(71) Applicant: Brainlab AG, Munich (DE)

(72) Inventors: Sebastian STOPP, Munich (DE); Marius LOCHER, Munich (DE); Valentin ELEFTERIU, Munich (DE)

(21) Appl. No.: 18/718,226

(22) PCT Filed: Dec. 17, 2021
(86) PCT No.: PCT/EP2021/086574

§ 371 Date: Jun. 10, 2024

(51) Int. Cl. A61B 34/20; G06T 7/13; G06T 7/30; G06T 7/73

(52) U.S. Cl. CPC A61B 34/20; G06T 7/13; G06T 7/30; G06T 7/73; A61B 2034/2055; A61B 2034/2065; G06T 2200/04; G06T 2207/10056; G06T 2207/20081; G06T 2207/30008

(57) ABSTRACT

The disclosed method relates to an approach of monitoring validity of a patient registration, wherein a contour of a rigid anatomical structure is initially determined in three-dimensional space, and its spatial position is constantly checked afterwards so as to detect a possible spatial deviation of the contour which may indicate an invalidated patient registration.

### FIELD OF THE INVENTION

The present invention relates to a computer-implemented method of monitoring validity of a patient registration, a corresponding computer program, a computer-readable storage medium storing such a program and a computer executing the program, as well as a medical system comprising an electronic data storage device and the aforementioned computer.

### TECHNICAL BACKGROUND

For guiding medical procedures, computer-assistant surgery regularly relies on a model of the patient the procedure is performed on, wherein the model is spatially registered, i.e. spatially aligned with the actual anatomy of the patient. For the success of such guided procedures, it is crucial that the patient model remains correctly registered for the entire duration of the procedure, so that medical appliances and instruments are correctly operated with respect to the patient's anatomy. Thus, it is an ongoing task for computer aided surgery to ensure that a patient registration remains valid.

A straightforward approach for doing so includes repeated registrations of the patient in predefined time intervals or after an incident has occurred that may have compromised the registration, which is however time consuming and error-prone.

WO 2014/117806 suggests an approach for determining whether or not a patient registration is still valid, which is based on two-dimensional image data. Registration errors that originate from positional deviations in a direction perpendicular to the image-plane however remain unrecognized.

The present invention has the object of providing a reliable and accurate approach of monitoring validity of a patient registration.

The present invention can be used for any kind of medical navigation procedures e.g. in connection with a medical navigation system such as Kick® and Curve®, both products of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The disclosed method relates to an approach of monitoring validity of a patient registration, wherein a contour of a rigid anatomical structure is initially determined in three-dimensional space, and its spatial position is constantly checked afterwards so as to detect a possible spatial deviation of the contour which may indicate an invalidated patient registration.

### GENERAL DESCRIPTION OF THE INVENTION

In this section, a description of the general features of the present invention is given for example by referring to possible embodiments of the invention.

In general, the invention reaches the aforementioned object by providing, in a first aspect, a computer-implemented medical method of monitoring validity of patient registration. The method comprises executing, on at least one processor of at least one computer (for example at least one computer being part of a navigation system), the following exemplary steps which are executed by the at least one processor.

In a (for example first) exemplary step, image data is acquired which describes a three-dimensional image dataset of a patient's body part. For example, such three-dimensional image dataset may be a CT-image dataset or a MRI-dataset from which a three-dimensional model of the patient's body part can be derived and subsequently registered with the actual body part of the patient.

In a (for example second) exemplary step, registration data is acquired which describes a positional registration of the patient's body part with the three-dimensional image dataset. Such registration may include a transformation matrix between a virtual image space with the model of the patient's body part, and the real world with the actual body part of the patient. For example, such transformation matrix may be created by identifying multiple landmarks of the patient's body and matching them to corresponding virtual landmarks of the model.

The registration thus acquired initially allows to superimpose the virtual image space including the three-dimensional image dataset and the model of the patient's body part derived therefrom, with the real world including the patient's actual anatomy and any medical instrument and appliances the spatial position thereof can be spatially tracked in the real world via a medical tracking system.

The approach described herein is to monitor validity of this initial registration, which is crucial for the superposition of the virtual image space and the real world.

Thus, in a (for example third) exemplary step, contour data is acquired which describes a contour of a rigid anatomical structure of the patient's body part within a three-dimensional co-ordinate system. In other words, the shape and position of a three-dimensional contour of a rigid, for example bony structure of the patient's body part is acquired. This may include acquiring a three-dimensional point-cloud on the contour's surface, which defines the contour's three-dimensional shape and from which the contour's three-dimensional shape can be extracted again at a later stage. For example, the contour is formed by a distinctive surface of the rigid or bony anatomical structure which can be easily identified by a practitioner. Said contour may be formed by a distinctive outline of, or a distinctive opening the anatomical structure. A surface scanner capable of determining the surface topography of objects may be utilised to acquire the point-cloud. In the alternative, any other suitable approach may utilise to acquire the point-cloud.

In a subsequent (for example fourth) exemplary step, observation data is acquired which describes a current spatial position of the contour. While this may include acquiring the shape of the entire contour, it may in some cases be sufficient to only acquire the spatial position of distinct features of the contour which may not only facilitate acquiring the observation data, but also the following step of determining possible deviations in the contour's spatial position.

In a (for example fifth) exemplary step, deviation data is determined based on the contour data, the registration data and the observation data, which describes a spatial relation between the initial spatial position described in the contour data and the current spatial position described in the observation data. In case the contour's spatial position described in the observation data does not deviate from the spatial position described in the contour data, it can be expected that the rigid or bony anatomical structure has not altered its position after the image dataset has been registered to the patient's body part, and that therefore the registration is still valid. On the other hand, a deviation in the contour's spatial position may indicate that the patient's body part has moved from its initial position and that the registration is therefore invalid.

In an example of the method according to the first aspect, the step of acquiring contour data includes acquiring raw contour data which describes a raw contour of the rigid anatomical structure of the patient's body part within a three-dimensional co-ordinate system, wherein the contour is obtained from matching the raw-contour to a reference defined in the three-dimensional image dataset.

This example includes an intermediate step where the shape of the contour is not directly determined, but is calculated from an associated contour of the rigid anatomical structure, that directly relates to the contour of the rigid anatomical structure. For example, if the contour to be determined is represented by an edge of a fracture or cut of a bone, the raw-contour may be represented by the fracture surface or the cut surface, respectively. In particular, the surfaces may lie adjacent to the edge of the fracture or the cut, respectively. In such case, the contour data needs to be derived from the raw contour data. For example, the raw-contour can be projected or otherwise matched onto a reference which is shown in the image data. In the above example, the acquired raw-contour of a cut surface is projected onto the bone surface seen in the three-dimensional image dataset, that includes the cutting edge. In a further example, this can be done by calculating, for each point of the determined point-cloud on the cut surface, the nearest point on the bone surface. The plurality of points calculated on the bone surface forms a point-cloud that eventually defines the cutting edge, i.e. the contour to be determined.

Any of the point-clouds defining the raw-contour or the contour can be determined via a surface-scanner which measures the distance between the scanner and a surface point, or via any other measure that is suitable to determine the spatial position of a point in three-dimensional space.

In a further example, the raw contour data describes the raw-contour, particularly the spatial position thereof, within a co-ordinate system assigned to a medical tracking system, for example the tracking system that is utilised to spatially track medical instruments or appliances with respect to the patient's anatomy.

In a further example, the contour data describes the contour, particularly the spatial position thereof within a co-ordinate system assigned to the image dataset and/or within a co-ordinate system assigned to the patient's body part.

In a further example, acquiring contour data includes identifying at least one distinctive feature of the contour, wherein
at least one distinctive feature is characterised by a local minimum and/or a local maximum in the contour's curvature; and/or wherein
an edge detection method is utilised, specifically by applying deep learning algorithms.

Distinctive features such as edges or other irregularities of the shape defined by the point-cloud, i.e. of the contour, allow for an improved detection and processing of the contour, which will be described further below. Those features can for example be defined by comparing the curvature or the surface normals along the contour, i.e. over neighbouring points of the point-cloud. Detected local minima or maxima may define specific features or edge points. Distinctive features of the contour may also be identified by implementing an edge detection approach known in the art, so as to identify edges in an image, that show discontinuities within the image, for example a significant change in the brightness of the image. Examples for such edge detection approaches which in particular may involve a deep learning algorithm are suggested by HU et al., (2020), “JSENet: Joint Semantic Segmentation and Edge Detection Network for 3D Point Clouds”, Hong Kong University of Science and Technology.

In a further example, the observation data is acquired via at least one optical camera of a surgical microscope and/or via a surface scanner of a surgical microscope, which is spatially tracked by the medical tracking system. For example, the contour and the spatial position thereof can be identified via a stereoscopic camera system. As described further above, this could also be accomplished via a surface scanner that measures, for each one of a plurality of points of the contour, the distance to the scanner. While such optical camera systems or surface scanner may be provided as independent, standalone systems, they may also be provided as integral components of a surgical microscope, which is for example utilized to visually observe a surgical site of the patient's body part.

Further, the step of determining deviation data may be based on at least one subset of the contour data describing the spatial position of at least one segment of the contour, particularly of at least one distinctive feature of the contour, the current position of which is described in the observation data.

If, for example, it is impossible for the camera system or the surface scanner to observe the entire contour, which may be the case when parts of the contour are visually obstructed by objects that have been brought between the contour and the camera system or the scanner, the suggested approach can be based on those parts of the contour which remain unobstructed for the camera system or the scanner. Preferably, those unobstructed parts comprise distinctive features as described above. Consequently, only those parts of the acquired contour data are processed, which find a counterpart in the observation data describing the visible, unobstructed parts of the contour.

Additionally or alternatively, determining deviation data may be based on observation data acquired in a plurality of spatial directions towards the rigid anatomical structure and/or at a plurality of points in time. For example, observation data that is acquired in different spatial directions towards the rigid anatomical structure and/or at a different points in time may describe the spatial position of different segments of the contour, particularly the spatial position of different distinctive features of the contour. Thus, if the observation data acquired via a single image frame is not sufficient to determine whether or not a spatial deviation of the contour has occurred, the observation data may be accumulated from a plurality of image frames. These image frames may be successively made, and/or may be made from different directions, so as to gather enough data that allows for determining whether or not the contour has moved with respect to its registered position.

Additionally or alternatively, determining deviation data is further based on additional data supplementing the observation data, wherein the additional data describes the contour or raw-contour, respectively. For example, those parts of the observation data which could not be determined via an optical camera or a surface scanner as described above, are “filled” with additional data acquired via other means, for example via a pointer instrument.

In case it has been determined that the contour has moved with respect to its initial registered position, indicating that the registration is invalid, the method according to the first aspect may further include the step of determining, based on the deviation data, deviation-characteristics data describing at least one characteristic of the spatial relation between the current spatial position described in the matching data and the spatial position described in the observation data, particularly at least one of the following characteristics:
the spatial position of a centre of rotation and/or the distance thereto;
the spatial position of an axis of rotation and/or the distance thereto;
the angle of rotation;
the spatial position and/or the direction of a translational shift;
the length of a translational shift;
wherein, based on the deviation-characteristics data, cause-analysis data is determined, which describes at least one plausible cause for the deviation, particularly wherein possible degrees of freedom of medical appliances are compared with the at least one characteristic of the spatial relation.

Generally speaking, possible positional deviations of the contour are analysed to identify plausible reasons that may have caused the positional deviation. This may be done by extracting translational and rotational components from the determined positional deviation. For example, a physical impact on the patient's body part or a tracking reference coupled to the patient's body part is expected to result in a translational deviation in the contour's position, whereas an unintended motion of the patient's body part within an immobilising structure, particularly of a patient's cranium within a head-clamp, is expected to result in a rotational deviation in the contour's position. Of course, such analysis may consider the physical properties of any structures that may have influence on the positional deviation, particularly possible degrees of freedom provided by these immobilising structures. This additional knowledge may help in identifying what structures may have failed and may have caused the positional deviation.

As long as a detected deviation in the contour's position is tolerable and does not compromise the medical proceedings, it may be left unconsidered with no further measures taken. Thus, a threshold value may be set for a tolerable deviation between the current spatial position described in the contour data and the spatial position described in the observation data. Further, output data may be determined based on the deviation data and the set threshold and, when the detected deviation exceeds the threshold, an alert signal and/or a request to repeat the step of acquiring registration data may be output. Thus, a user is instantly informed of an intolerable deviation in the contour's position. Further, the user may also be informed of possible reasons which may have caused this deviation.

In a specific example of the method according to the first aspect, the patient's body part is a cranial bone of a patient and the rigid anatomical structure is defined by an aperture created in the cranial bone for performing craniotomy.

In a second aspect, the invention is directed to a computer program comprising instructions which, when the program is executed by at least one computer, causes the at least one computer to carry out method according to the first aspect. The invention may alternatively or additionally relate to a (physical, for example electrical, for example technically generated) signal wave, for example a digital signal wave, such as an electromagnetic carrier wave carrying information which represents the program, for example the aforementioned program, which for example comprises code means which are adapted to perform any or all of the steps of the method according to the first aspect. The signal wave is in one example a data carrier signal carrying the aforementioned computer program. A computer program stored on a disc is a data file, and when the file is read out and transmitted it becomes a data stream for example in the form of a (physical, for example electrical, for example technically generated) signal. The signal can be implemented as the signal wave, for example as the electromagnetic carrier wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention according to the second aspect therefore may alternatively or additionally relate to a data stream representative of the aforementioned program, i.e. comprising the program.

In a third aspect, the invention is directed to a computer-readable storage medium on which the program according to the second aspect is stored. The program storage medium is for example non-transitory.

In a fourth aspect, the invention is directed to at least one computer (for example, a computer), comprising at least one processor (for example, a processor), wherein the program according to the second aspect is executed by the processor, or wherein the at least one computer comprises the computer-readable storage medium according to the third aspect.

In a fifth aspect, the invention is directed to a medical system, comprising:
a) the at least one computer according to the fourth aspect;
b) at least one electronic data storage device storing at least the image data; and
c) a medical device for carrying out a medical procedure on the patient, wherein the at least one computer is operably coupled to
   the at least one electronic data storage device for acquiring, from the at least one data storage device, at least the image data, and
   the medical device for issuing a control signal to the medical device for controlling the operation of the medical device on the basis of the deviation data.

Alternatively or additionally, the invention according to the fifth aspect is directed to a for example non-transitory computer-readable program storage medium storing a program for causing the computer according to the fourth aspect to execute the data processing steps of the method according to the first aspect.

For example, the invention does not involve or in particular comprise or encompass an invasive step which would represent a substantial physical interference with the body requiring professional medical expertise to be carried out and entailing a substantial health risk even when carried out with the required professional care and expertise.

### Definitions

In this section, definitions for specific terminology used in this disclosure are offered which also form part of the present disclosure.

The method in accordance with the invention is for example a computer-implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term computer includes a server resource. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a computer program comprising instructions which, when on the program is executed by a computer, cause the computer to carry out the method or methods, for example, the steps of the method or methods, described herein and/or to a computer-readable storage medium (for example, a non-transitory computer-readable storage medium) on which the program is stored and/or to a computer comprising said program storage medium and/or to a (physical, for example electrical, for example technically generated) signal wave, for example a digital signal wave, such as an electromagnetic carrier wave carrying information which represents the program, for example the aforementioned program, which for example comprises code means which are adapted to perform any or all of the method steps described herein. The signal wave is in one example a data carrier signal carrying the aforementioned computer program. The invention also relates to a computer comprising at least one processor and/or the aforementioned computer-readable storage medium and for example a memory, wherein the program is executed by the processor.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

The n-dimensional image of a body is registered when the spatial location of each point of an actual object within a space, for example a body part in an operating theatre, is assigned an image data point of an image (CT, MR, etc.) stored in a navigation system.

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

A pointer is a rod which comprises one or more-advantageously, two-markers fastened to it and which can be used to measure off individual co-ordinates, for example spatial co-ordinates (i.e. three-dimensional co-ordinates), particularly of one or more points of a point-cloud, on a part of the body, wherein a user guides the pointer (for example, a part of the pointer which has a defined and advantageously fixed position with respect to the at least one marker attached to the pointer) to the position corresponding to the co-ordinates, such that the position of the pointer can be determined by using a surgical navigation system to detect the marker on the pointer. The relative location between the markers of the pointer and the part of the pointer used to measure off co-ordinates (for example, the tip of the pointer) is for example known. The surgical navigation system then enables the location (of the three-dimensional co-ordinates) to be assigned to a predetermined body structure, wherein the assignment can be made automatically or by user intervention.

The present invention is also directed to a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

The invention also relates to a navigation system for computer-assisted surgery, comprising:
a computer for processing the absolute point data and the relative point data;
a detection device for detecting the position of the main and auxiliary points in order to generate the absolute point data and to supply the absolute point data to the computer;
a data interface for receiving the relative point data and for supplying the relative point data to the computer; and
a user interface for receiving data from the computer in order to provide information to the user, wherein the received data are generated by the computer on the basis of the results of the processing performed by the computer.

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia.

The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates the basic steps of the method according to the first aspect;

FIG. 2 schematically shows a medical set-up utilising the present invention, specifically the method according to the first aspect;

FIG. 3 is a close-up of FIG. 2.

### DESCRIPTION OF EMBODIMENTS

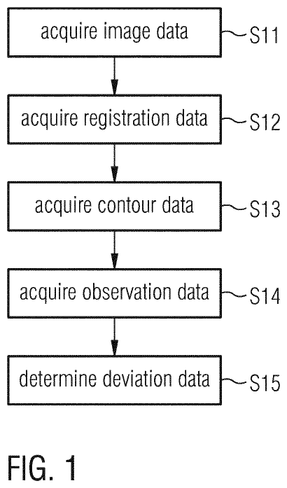

FIG. 1 illustrates the basic steps of the method according to the first aspect, in which step S11 encompasses acquiring image data, step S12 encompasses acquiring registration data, step S13 encompasses acquiring contour data, step S14 encompasses acquiring observation data and subsequent S15 encompasses determining deviation data.

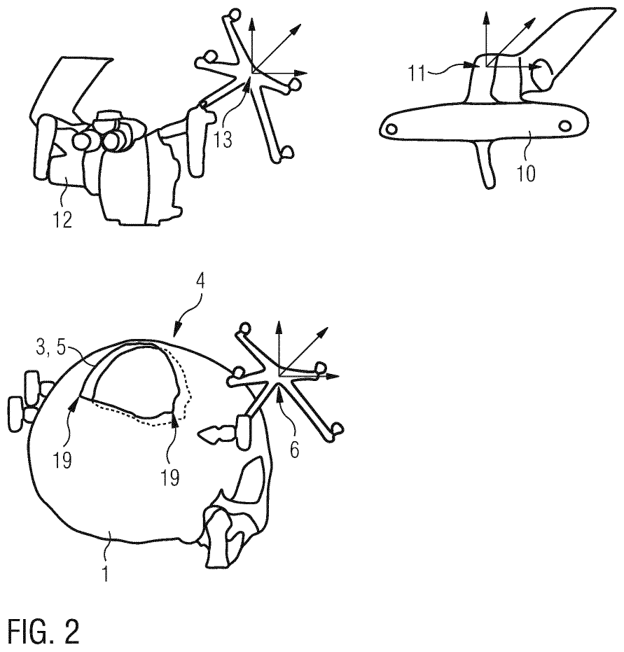

FIG. 2 schematically shows a common medical set-up used for craniotomy, for which a section of the cranial bone is removed so as to gain access to the brain. In the shown example, the patient's cranium 1 is immobilised via a head-clamp (not indicated) having a reference star which defines a co-ordinate system 6 assigned to the patient's cranium 1, and which therefore needs to remain static with respect to the patient's cranium 1.

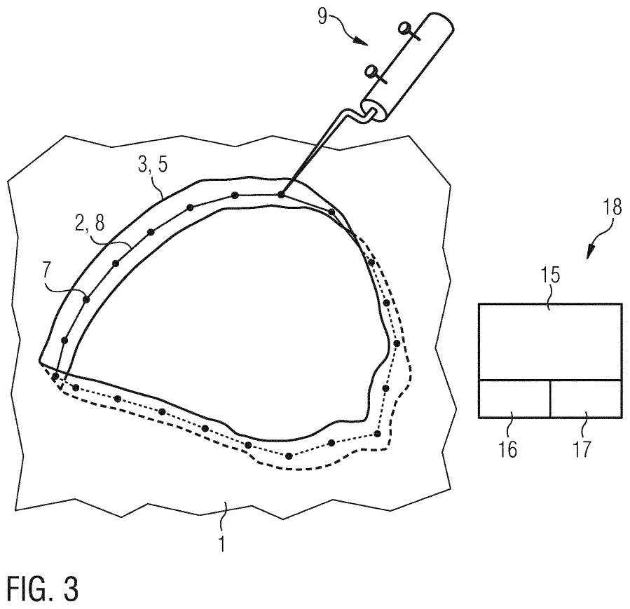

A surgical microscope 12 for observing the surgical site is also provided with a reference star that defines a co-ordinate system 13 assigned to the surgical microscope. The reference stars are fixedly attached to the cranial bone 1 and the surgical microscope 12, respectively, and are positionally tracked via a medical tracking system 10 that features a stereoscopic camera array and transmits the video image obtained via these two cameras to a medical navigation system 18 (FIG. 3). The medical navigation system 18 comprises a computer 15 having a data storage 16 and a processor 17 for processing the video images obtained from the tracking system 10. The positions of co-ordinate systems 6 and 13 relative to the co-ordinate system 11 assigned to the tracking system are calculated from the received images via triangulation. Registering the cranial bone 1 with respect to the co-ordinate system 6 and registering the optics of microscope 12 with respect to co-ordinate system 13 enables the medical navigation system 18 to determine the relative position of these features.

The aperture 4 may be registered via a surface scanner or by tracing or palpating the circumferential cutting surface 2 with a tracked pointer instrument 9 (FIG. 3), thereby determining the spatial position of a plurality of points that form a three-dimensional point-cloud 8. Projecting each one of the points to the nearest location on the outer surface 5 of the cranial bone 1 defines the contour 3 of the aperture 4. In the example shown in FIG. 2, the contour 3 includes two distinctive sections where the curvature of the contour 3 significantly differs from the curvature of the remaining contour 3, thereby forming two distinctive features 19 in the contour 3. As these distinctive features 19 can be easily identified, the following approach of monitoring validity of the initial registration may be entirely based on tracking the spatial position of these features 19 over time.

Once the shape and the position of the contour 3 has been initially determined within the co-ordinate system 6 and/or within the co-ordinate system 11, any deviation from this initial position at a later point in time may indicate an unintended motion of the cranial bone 1 within the immobilising head-clamp, or an unintended motion of the reference star defining co-ordinate system 6 with respect to the head-clamp.

After the initial position of the aperture 4 has been determined, its position may further be supervised via a surface scanner (not shown) of the surgical microscope 12. In case such positional deviation of the aperture 4 has been detected, the medical navigation system 18 outputs an audible and/or visible alarm signal informing about a possible invalidity of the initial registration of the cranial bone 1. The initial registration may then be restored by re-determining the spatial position of the aperture 4 within the co-ordinate system 6. In the alternative, the initial registration may be restored by applying the three-dimensional transformation matrix between the initially determined position of the aperture 4 and the current position of the aperture 4 so as to base the further proceedings on the current position of the aperture 4.

1. A computer-implemented method of monitoring validity of a patient registration, the method comprising:
acquiring image data that describes a three-dimensional image dataset of a body part of a patient;
acquiring registration data that describes a positional registration of the body part of the patient with the three-dimensional image dataset;
acquiring contour data that describes a spatial position of a contour of a rigid anatomical structure of the body part of the patient within;
a three-dimensional co-ordinate system assigned to the three-dimensional image dataset of the body part of the patient; and/or
a three-dimensional coordinate system assigned to the body part of the patient;
acquiring observation data via an associated surgical microscope that is spatially tracked via an associated medical tracking system, wherein the observation data describes a current spatial position of the contour within a coordinate system assigned to an optical camera and/or a scanner of the associated surgical microscope; and
determining deviation data based on the contour data, the registration data and the observation data, wherein the deviation data describes a spatial relation between the spatial position of the contour described in the contour data and the current spatial position of the contour described in the observation data.

2. The method according to claim 1, wherein the acquiring the contour data comprises acquiring raw-contour data that describes a raw-contour of the rigid anatomical structure of the body part of the patient within a three-dimensional co-ordinate system, wherein the contour is obtained from matching the raw-contour to a reference defined in the three-dimensional image dataset.

3. The method according to claim 2, wherein:
the acquiring the raw-contour data comprises acquiring three-dimensional positions of a plurality of points defining a point-cloud; and/or
the acquiring the raw-contour data comprises acquiring the raw-contour data via a pointer instrument that is spatially tracked via the associated medical tracking system.

4. The method according to claim 2, wherein the acquiring the raw-contour data comprises acquiring raw-contour data that describes the spatial position of the raw-contour within a co-ordinate system assigned to the associated medical tracking system.

5. (canceled)

6. The method according to claim 4, wherein the acquiring the contour data comprises acquiring contour data that identifies at least one distinctive feature of the contour, wherein:
the at least one distinctive feature is characterized by a local minimum and/or a local maximum in a curvature of the contour; and/or
the at least one distinctive feature is identified using an edge detection method by applying deep learning algorithms.

7. (canceled)

8. (canceled)

9. The method according to claim 1, wherein the determining the deviation data comprises determining the deviation data based on at least one subset of the contour data describing the spatial position of at least one distinctive feature of the contour, wherein the current position of the at least one distinctive feature of the contour is described in the observation data.

10. The method according to claim 1, wherein the determining the deviation data comprises determining the deviation data based on observation data acquired in a plurality of spatial directions towards the rigid anatomical structure and/or at a plurality of points in time, wherein the observation data is acquired in different spatial directions towards the rigid anatomical structure and/or at a different points in time describing the spatial position of different distinctive features of the contour.

11. The method according to claim 3, wherein the determining the deviation data comprises determining the deviation data based on additional contour data and/or additional raw-contour data supplementing the observation data, wherein the additional contour data and/or additional raw-contour data describes the contour or raw-contour, respectively, wherein the acquiring the additional contour data and/or additional raw-contour data comprises acquiring the three-dimensional position of an additional plurality of points defining a point-cloud and/or wherein the additional-raw-contour data is acquired via the pointer instrument and/or wherein the additional raw-contour is matched to the reference (5) defined in the three-dimensional image dataset.

12. The method according to claim 1, further comprising:
determining, based on the deviation data, deviation-characteristics data describing at least one characteristic of the spatial relation between the current spatial position described in the matching data and the spatial position described in the observation data, wherein the at least one characteristic comprises at least one of:
a spatial position of a centre of rotation and/or the distance thereto;
a spatial position of an axis of rotation and/or the distance thereto;
an angle of rotation;
a spatial position and/or the direction of a translational shift; and/or
a length of a translational shift; and
determining cause-analysis data based on the deviation-characteristics data, wherein the cause-analysis data describes at least one plausible cause for the deviation, wherein possible degrees of freedom of medical appliances are compared with the at least one characteristic of the spatial relation.

13. The method according to claim 1, further comprising:
setting a threshold value for a tolerable deviation between the current spatial position described in the contour data and the spatial position described in the observation data; and
determining output data based on the detected deviation exceeding the threshold, wherein the output data describes an alert signal and/or a request to repeat the acquiring registration data.

14. The method according to claim 1, wherein the body part of the patient comprises a cranial bone of the patient and the rigid anatomical structure comprises aperture created in the cranial bone for performing craniotomy.

15. A non-transient computer-readable storage medium storing instructions thereon that, when the instructions are executed by a processor of an associated computer cause the computer to perform a method of monitoring validity of a patient registration, comprising:
acquiring image data that describes a three-dimensional image dataset of a body part of a patient;
acquiring registration data that describes a positional registration of the body part of the patient with the three-dimensional image dataset;
acquiring contour data that describes a spatial position of a contour of a rigid anatomical structure of the body part of the patient within:
a three-dimensional co-ordinate system assigned to the three-dimensional image dataset of the body part of the patient; and/or
a three-dimensional coordinate system assigned to the body part of the patient;
acquiring observation data via an associated surgical microscope that is spatially tracked via an associated medical tracking system, wherein the observation data describes a current spatial position of the contour within a coordinate system assigned to an optical camera and/or a scanner of the associated surgical microscope; and
determining deviation data based on the contour data, the registration data and the observation data, wherein the deviation data describes a spatial relation between the spatial position of the contour described in the contour data and the current spatial position of the contour described in the observation data.

16. A medical system, comprising:
at least one computer comprising a processor and a non-transient computer-readable storage medium storing instructions thereon that are executable by the processor;
at least one electronic data storage device storing image data; and
a medical device operable to carry out a medical procedure on a patient,
wherein the processor is operable to execute the instructions stored on the non-transient computer-readable storage medium to perform a method of monitoring validity of a patient registration by:
acquiring the image data that describes a three-dimensional image dataset of a body part of a patient;
acquiring registration data that describes a positional registration of the body part of the patient with the three-dimensional image dataset;
acquiring contour data that describes a spatial position of a contour of a rigid anatomical structure of the body part of the patient within:
a three-dimensional co-ordinate system assigned to the three-dimensional image dataset of the body part of the patient; and/or
a three-dimensional coordinate system assigned to the body part of the patient;
acquiring observation data via an associated surgical microscope that is spatially tracked via an associated medical tracking system, wherein the observation data describes a current spatial position of the contour within a coordinate system assigned to an optical camera and/or a scanner of the associated surgical microscope; and
determining deviation data based on the contour data, the registration data and the observation data, wherein the deviation data describes a spatial relation between the spatial position of the contour described in the contour data and the current spatial position of the contour described in the observation data,
wherein the at least one computer is operably coupled to:
the at least one electronic data storage device for acquiring the image data from the at Least one electronic data storage device, and
the medical device for issuing a control signal to the medical device for controlling operation of the medical device based on the deviation data.
